(19) United States
(12) Patent Application Publication
(10) Pub. No.: US 2026/0273270 A1
(43) Pub. Date: Sep. 17, 2026

(54) PAINTABLE AND SELF-ADHESIVE HYDROGEL ELECTRODES

(71) Applicant: THE GOVERNING COUNCIL OF THE UNIVERSITY OF TORONTO, Toronto (CA)

(72) Inventors: Jia Xi CHEN, Toronto (CA); Zia SAADATNIA, North York (CA); Milos POPOVIC, Mississauga (CA); Hani NAGUIB, Toronto (CA)

(21) Appl. No.: 19/163,196

(22) PCT Filed: Mar. 7, 2024
(86) PCT No.: PCT/CA2024/050282

§ 371 Date: Sep. 8, 2025

Related U.S. Application Data
(60) Provisional application No. 63/450,721, filed on Mar. 8, 2023.

(51) Int. Cl. A61N 1/04; C09J 139/04; C09J 199/00

(52) U.S. Cl. CPC A61N 1/0496; C09J 139/04; C09J 199/00

(57) ABSTRACT

A self-adhesive, conductive hydrogel composition for use as a skin-contacting electrode comprises a hydrogel comprising a crosslinked hydrophilic matrix polymer, a conductive agent, and an adhesive agent. The conductive and adhesive agents may comprise up to about 5% w/w of the composition. The hydrogel may be carboxymethyl cellulose with phytic acid as crosslinker; the conductive agent may be polystyrene sulfonate; and the adhesive agent may comprise tannic acid, polydopamine, or a combination of both. The composition may be prepared from a mixture of a first precursor of the hydrophilic matrix polymer or a salt thereof with water and the adhesive and conductive agents and a second precursor with the crosslinking agent.

### CROSS-REFERENCE TO RELATED APPLICATIONS

This application claims priority to and the benefit of U.S. Provisional Application No. 63/450,721 filed Mar. 8, 2023, the entirety of which is incorporated herein by reference.

### TECHNICAL FIELD

The present disclosure relates to hydrogel compositions suitable for use as electrodes in electrical stimulation therapy, and electrodes formed of such compositions.

### TECHNICAL BACKGROUND

Surface functional electrical stimulation (sFES) is the process of inducing contractions in paralyzed muscles through the delivery of electric currents via non-invasive electrodes. Aside from directly modulating muscles to instantaneously recover lost motor functions, sFES has been found effective in improving voluntary contractions for patients paralyzed by injury, stroke, or neurological diseases. As a treatment modality, sFES is applicable to arbitrary sites of the body but is a labor-intensive and time-consuming method since it is conventionally provided in a clinical setting. Due to the large variance in muscle distribution among different individuals, commercially available stimulation electrodes of predefined shapes and sizes must be applied by a clinician to ensure the selectivity and precision of sFES-activated muscles.

Meanwhile, paintable and injectable conductive hydrogels have been shown to have potential in medical applications. For example, poly(3,4-ethylenedioxythiophene) polystyrene sulfonate (PEDOT:PSS) has been fabricated in aldehyde hyaluronic acid, glycol chitosan, and polyethylene glycol for cell cultures. For repair of the heart organ, pyrrole groups and dopamine groups have been incorporated in a gelatin hydrogel as injectable cardiac patches while PEDOT:PSS has been dispersed in collagen and alginate matrices as engineered cardiac tissue. In skin applications, PEDOT:PSS and guar slime have been made into a hydrogel; alternative approaches to injectable hydrogel have included with silver nanoparticles, polyaniline, and polyvinyl alcohol for wound dressings.

### BRIEF DESCRIPTION OF THE DRAWINGS

In drawings which illustrate by way of example only embodiments of the present invention,

FIG. 1 is a graph of Fourier-transform infrared spectroscopy (FTIR) spectra of an example hydrogel composition.

FIG. 2 depicts scanning electron microscope (SEM) images of the lyophilized hydrogel composition.

FIG. 3 is a graph illustrating the effect of phytic acid concentration on viscosity in sample of the example hydrogel composition.

FIG. 4 is a graph illustrating the adhesion strength of an example hydrogel composition on different substrates.

FIG. 5 is a graph illustrating a transient conductivity property of an example hydrogel composition.

FIG. 6 is a Bode plot comparing the impedance of different materials as stimulation electrodes, including the example hydrogel composition.

FIG. 7 is a photograph showing extrusion of an example hydrogel composition from a syringe.

FIG. 8 is a photograph showing the extruded example hydrogel composition applied to a human arm.

FIG. 9 is a series of photographs comparing the use of commercially available electrodes with electrodes formed from the example hydrogel composition.

FIG. 10 is an illustration of an example dispensing apparatus for the hydrogel composition.

### DETAILED DESCRIPTION

Soft conductive materials have been found potentially useful in wearable applications because of their ability to conform to the human skin. However, variabilities in muscle distribution from person to person pose challenges in developing wearable and personalized health care solutions, such as surface functional electrical stimulation (sFES) systems.

As mentioned above, sFES is the process of inducing contractions in paralyzed muscles via electric currents, which are delivered by electrodes placed on the skin. Commercially available stimulation electrodes are provided in predefined shapes and sizes, since customizability is generally not feasible at a commercial scale. Consequently, sFES is typically administered in a clinical setting, and requires the precise application of the commercially available electrodes to ensure that the current is delivered to the appropriate muscles. Therefore, a skin-adhesive and conductive material that can be conveniently applied directly to the skin, for example by painting on the skin, would provide a facile approach to achieving self-applied and home-based sFES sessions. While customizable and self-applicable electrodes have been developed, to date, most are for sensing functionality or perception stimulation purposes, rather than for transmitting the relatively higher current needed to contract muscles.

Accordingly, as set out in the examples and embodiments described herein, this disclosure provides a novel hydrogel composition for electrodes that are adaptable to be of any shape and size, with self-adhesive properties to enable the electrodes to conform to body morphology and provide stimulation signal stability; electrodes formed from such compositions, and methods and apparatuses for applying the composition to a subject's skin for use as electrodes. The composition may comprise naturally occurring, biocompatible, and eco-friendly biopolymers and are easy to fabricate and use as stimulation electrodes for the bioelectronic interface to establish sFES sessions with better efficiency and accuracy than commercially available electrodes of predefined size and shape.

These hydrogel composition comprises a skin-compatible hydrophilic polymer, with at least one conductive agent and at least one adhesive agent. The viscosity of the composition may be adjusted by the inclusion of at least one crosslinking agent to produce a composition with appropriate viscosity, and thus shear strength, suitable for depositing on human skin (e.g., by painting the composition directly on the skin surface) and retaining a lead wire or other conductor in place. The adhesive agent enhances the composition's adhesion to human skin without the need for additional adhesives; thus, the composition may be considered self-adhesive.

In the example formulation described below, the hydrophilic matrix polymer is a polysaccharide, and specifically the cellulose derivative carboxymethyl cellulose (CMC). CMC is selected in the example below because it is considered to be biocompatible and generally hypoallergenic. Other polymers suitable for use as the matrix providing the main network structure in the hydrogel composition include polyacrylamide (PAM), collagen, poly(N-isopropylacrylamide) PNIPAM, polypyrrole (PPy), poly(vinyl alcohol) (PVA), guar gum, glycerol, hyaluronic acid, agarose, poly(acrylic acid) (PAAc), lignin, other forms of the derivatives of the polymers identified above, as well as arbitrary mixtures of two or more of the polymers identified above.

The conductive agent in the example formulation comprises poly(3,4-ethylenedioxythiophene) polystyrene sulfonate (PEDOT:PSS), a mixture of ionomers known for its ionic and electronic dual-mode conductivity and its biocompatibility. Other suitable conductive agents can include polyaniline (PANi), PPy, polythiophene (PTh), graphene oxide (GO), multiwalled carbon nanotube (CNT), metal ions such as lithium chloride (LiCl) and iron (III) chloride (FeCl₃), as well as in other forms such as silver nanoparticles (Ag NPs).

The adhesive agent in the example formulation is tannic acid, a naturally occurring polyphenol known for good affinity with skin and other substrates. Other suitable adhesive agents can include the mussel-inspired bio-adhesive polydopamine, cyanoacrylate-based products such as methyl-2-cyanoacrylate, n-butyl-2-cyanoacrylate, gelatin-based products such as resorcinol and formaldehyde or gelatin and glutaraldehyde, polyethylene glycol (PEG)-based products, and albumin and glutaraldehyde-based products.

The crosslinking agent in the example formulation comprises phytic acid, a naturally occurring physical crosslinking agent. Other suitable crosslinking agents can include epichlorohydrin (ECH), aldehyde-based reagents, urea derivatives, metallic ions such as zinc, nickel, iron, copper, silver, gold, cobalt, molybdenum, antimony, lead, calcium, sodium, potassium, arsenic, and mercury.

The conductive, adhesive, and crosslinking agents may provide additional functionality in the hydrogel composition. For instance, the adhesive agent in the example formulation below, tannic acid, also has crosslinking ability and can function as a physical or chemical crosslinker in a polymeric network. The selected crosslinking agent, phytic acid, may contribute to the conductivity of the final hydrogel composition due to the introduction of chemical functional groups as hydrogen bond donors and anionic groups in electrostatic attractions, as well as the generation of free hydrogen ions in water and its resultant partial ionization. The effect of these agents on the characteristics of the final composition will depend on their concentration, pH, and temperature during the manufacturing process. In this disclosure, these constituents are described according to their primary function as the adhesive, crosslinking, and conductive agent, as the case may be, but it will be understood by those skilled in the art that these agents may also enhance the other characteristics of the composition. Further, while the examples and embodiments discussed below refer to only one hydrophilic matrix polymer as the main structure of the network, conductive agent, adhesive agent, and crosslinking agent, those skilled in the art will appreciate that more than one hydrophilic matrix polymer, conductive agent, adhesive agent, and crosslinking agent may be employed. References to “a” or “the” hydrophilic matrix polymer, conductive agent, adhesive agent, or crosslinking agent include combinations of more than one such constituent unless the context indicates otherwise.

Preparation of the hydrogel composition may proceed in two principal phases. In the first phase, the adhesive agent and/or the conductive agent are introduced to the hydrophilic matrix polymer or the crosslinking agent. In the example formulation, the adhesive and conductive agents, tannic acid and PEDOT:PSS, are combined with the hydrophilic matrix polymer in a first precursor, and the crosslinking agent, PA, comprises second precursor. In the second phase, the two precursors are mixed to produce the final hydrogel composition for application as an electrode. Typically, the two precursors are mixed only shortly before applying the hydrogel composition, since gelation of the hydrogel composition upon exposure to air may occur quickly, within a time window of minutes.

In one embodiment, the first precursor comprises the hydrophilic matrix polymer, adhesive agent, and conductive agent. The first precursor is subsequently mixed with the second precursor, which may contain only the crosslinking agent. Both the first and second precursors may comprise a suitable aqueous medium (typically, water) to promote miscibility when the precursors are combined, so that the crosslinking agent may distributed substantially evenly throughout the hydrogel composition.

When used as an electrode, the hydrogel composition may be applied directly to a patient's skin, for example by means of a paintbrush, sponge, spatula, extruder, or other suitable applicator. In accordance with the example discussed below, the applicator may be a dispensing apparatus such as a syringe. Since the application of the hydrogel composition involves or resembles painting the skin with the composition, the composition effectively fills the crevices and hair follicles on the skin surface, improving contact. A conductor (e.g., a lead wire) can then be adhered to the applied composition, optionally coated with additional composition. After gelation, the conductor remains in place and may be connected to an electrical stimulator device, or indeed any other electrical device (e.g., electrocardiogram). The conductor may be provided pre-coated with the hydrogel composition to facilitate adhesion to the freshly applied composition on the patient's skin and crosslinking to the hydrogel electrodes. The pre-coating may be done during the therapy session, or earlier in advance, so that the hydrogel composition on the conductor has completely gelled.

For convenience in dispensing the hydrogel composition, particularly for self-administration by a patient, the first and second precursors may be packaged separately and distributed in a kit or package, optionally together with a suitable applicator. The precursors may be mixed in any suitable container, and then applied using the applicator.

Alternatively, the first and second precursors may be packaged within the dispensing apparatus in discrete chambers, as in a double-barreled or two-part syringe, tube, or the like. The dispensing apparatus may include a mixing chamber in fluid communication with the chambers holding the first and second precursors. In use, the precursors are allowed to enter the mixing chamber, where the two precursors are mechanically mixed, then dispensed through a nozzle or other aperture in fluid communication with the mixing chamber.

### Fabrication of Example Hydrogel Compositions

An example hydrogel composition was fabricated with CMC. Initially, 0.7 g of carboxymethyl cellulose sodium salt was diluted by 10 mL of deionized (DI) water under vigorous magnetic stirring for 24 h. Then, the 7% CMC solution was heated to 60° C., and 5 g of tannic acid (TA) was added. After mechanical mixing for 3 h and cooling to room temperature, 5 mL of PEDOT:PSS (1.0 wt % in water) was added into the TA-CMC solution. Various amounts of phytic acid (PA, 50 wt % in water) as listed in Table 1 was mixed in with the TA-PEDOT-CMC solution to prepare a highly viscous pre-gel solution (i.e., the hydrogel composition prior to gelation). The ratio of PA to CMC weight ranged from 30% (PA30-TA-PEDOT-CMC) to 80% PA80-TA-PEDOT-CMC). The materials selected for these examples are advantageous because they are naturally occurring or easily derivable from natural sources, biodegradable, and of course suitable for contact with human skin.

Gelation occurred within minutes of exposure of the mixture to air at room temperature.

TABLE 1
Example hydrogel compositions.

| | PA |
|---|---|
| Sample Code | (mL) |
| PA30-TA-PEDOT-CMC | 1.467 |
| PA40-TA-PEDOT-CMC | 1.955 |
| PA50-TA-PEDOT-CMC | 2.444 |
| PA60-TA-PEDOT-CMC | 2.933 |
| PA70-TA-PEDOT-CMC | 3.422 |
| PA80-TA-PEDOT-CMC | 3.911 |

The amount of CMC, TA, and PEDOT:PSS was fixed as described above.

### Characterization of Example Hydrogel Compositions

The chemical structures of the hydrogel composition (post-gelation) and the pre-gel solution were determined by placing the sample in ambient conditions on an FTIR spectrometer (ALPHA II, Bruker), with the obtained spectra being an average of 20 scans. To probe the morphology, the hydrogel compositions were first cooled to cryogenic temperatures with liquid nitrogen, lyophilized, and cooled to cryogenic temperature again to be fractured into cross-sections. The morphology was studied with a scanning electron microscope (SEM, JSM-IT100, InTouchScope). To quantify paintability, the pre-gel solutions were measured with a rheometer (MCR702e, Anton-Paar). For the adhesion properties, lap shear strength testing was conducted with the hydrogel composition sandwiched between various substrates, under exerted shear force from a micro tester (Instron 5848, Aaron Equipment). Electrical properties were measured with the four-point probe method (Keithley 2400, Tektronix) for conductivity, while impedance was determined with the hydrogel composition in contact with porcine trotters under an applied voltage with electrochemical instrumentation (CHI6056, CH Instruments). For comparisons across different electrode materials, the contact areas were restricted to be less than the dimension 2 cm². Lastly, the closure of the eye was induced by current pulses of 0.4 ms duration at 40 Hz frequency, outputted from a single channel of an 8-channel stimulator (MyndMove, MyndTec) for sFES purposes.

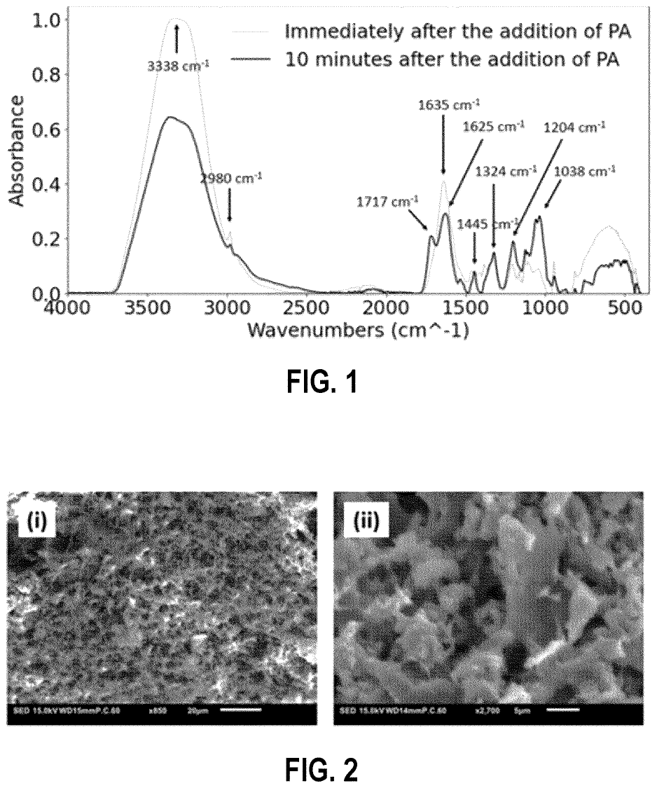

The pre-gel solution (immediately after the addition of PA into the TA-PEDOT-CMC solution) was compared with the hydrogel composition after gelation in air for 10 minutes. It was found that were no observable differences in the absorption bands across FTIR spectra for hydrogel compositions of various PA concentrations. FIG. 1 depicts the FTIR spectra. The presence of CMC contributed to the broad absorption band at 3338 cm⁻¹, due to the vibration frequency of the —OH groups, the small peak at 1445 cm⁻¹, due to the bending frequency of the C—H methyl groups, and the prominent peak at 1038 cm⁻¹, due to the stretching of C—O bonds.

The vibration of —OH groups in TA was found to contribute to the 3338 cm⁻¹ band, while bending of the —OH groups on the phenol rings of TA is responsible for the absorbance bands at 1324 cm⁻¹. The peak at 1204 cm⁻¹ was attributed to the P—O—C bonds in PA. The subtle peak at 2980 cm⁻¹ was found to characterize the stretching vibration of C—H groups. After the gelation process, however, the prominent band at 1635 cm⁻¹ due to P═O bonds stretching, representative of the PA spectrum, was shifted to 1625 cm⁻¹ after the formation of the solid PCAC hydrogel due to its participation in hydrogen bonding, leading to a mechanically stable structure on the macroscopic level. The appearance of the new 1717 cm⁻¹ band in PCAC hydrogel indicated the formation of COOH, or carboxylic acid groups since the introduction of PA encouraged the substitution of Na+ in CMC polymer chains with the new ion donor of H+ in PA. Without wishing to be bound by theory, it was concluded from the FTIR spectra that PA promotes the formation of hydrogen bonds, giving rise to a stable structure among compounds.

The morphology of the hydrogel composition was studied with SEM. Since no visual difference was observed among various PA concentrations, FIG. 2 shows the morphology of PA70-TA-PEDOT-CMC after the gelation process at (i) 850× and (ii) 2700× magnification. Although changes in pore size during lyophilization might occur due to the expansion in the volume of water, it was concluded from the SEM images that the final hydrogel composition has a substantially even distribution of open-cell pores. It is believed that both the uniformity in distribution and the open cell structure permits water within the hydrogel to flow freely during stimulation, reducing the occurrence of local spikes in conductivity which may lead to locally concentrated electric fields and skin burns during stimulation. Thus, the hydrogel solution is advantageous for stimulation purposes when in contact with the skin.

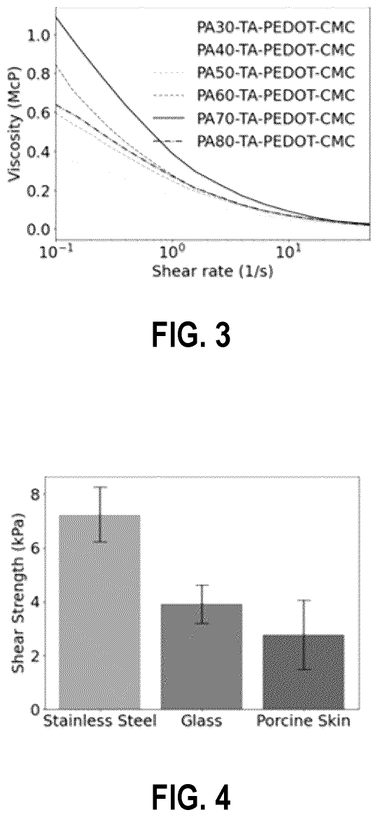

To establish paintability, the pre-gel solutions were extruded through a syringe with a 1.5 mm diameter opening. PA concentration was examined with respect to the viscosity of the resulting pre-gel samples. FIG. 3 shows the measured material properties against PA concentrations, varied according to Table 1. It was found that a ratio of PA to CMC weight below about 30% had low viscosity, leading to immediate spreading outside of a confined region once painted, and that a ratio over about 80% resulted in immediate gelation leading to an overall inhomogeneous texture. All pre-gel solutions depicted in FIG. 3 exhibited the shear thinning behavior of decreasing viscosity under shear strain, which is common among non-Newtonian fluids, indicating that the solution demonstrates flow property ideal for extrusion. Furthermore, an intuitively proportional relationship between increasing PA concentration and increasing viscosity was observed, as the addition of PA encourages the formation of hydrogen bonds. However, for the PA80-TA-PEDOT-CMC sample, its viscosity decreased to be similar to the PA50-TA-PEDOT-CMC sample. Without wishing to be bound by theory, this may be due to the addition of PA above a certain threshold leading to immediate gelation in local areas before mechanical stirring can evenly disperse PA throughout the entire solution. Because chunks of the hydrogel composition underwent the gelation process before the rest of the solution, the viscosity decreased as the effective concentration of PA dropped locally in the pre-gel solution. For application as paintable stimulation electrodes, high viscosity is desirable because it minimizes the spread of extruded lines before the gelation process, allowing for higher precision and accuracy in controlling the effective area of a bioelectronic interface. Thus, of the depicted samples, the PA70-TA-PEDOT-CMC sample (ratio of PA to CMC weight of 70%) was found to be particularly suitable for sFES purposes due to its high selectivity in stimulated muscles, enabled by high viscosity. Additionally, it was also found that any concentration of PEDOT:PSS in water, from about 1 (as in the examples above) to 100% (i.e., no water), was suitable for use in the compositions.

FIG. 4 illustrates the adhesive strength of the PA70-TA-PEDOT-CMC sample with various substrates, tested by the lap shear adhesion test with shear strength determined by the maximum force over the contact area of the hydrogel with respective substrates. The error bars show the standard deviation of seven samples. All samples characterized in FIG. 4 were made from biocompatible components, with pH levels of the hydrogel composition at 4-5. The lap shear adhesion test was employed since sFES electrodes are typically placed onto the skin vertically with respect to the ground. When used as sFES electrodes, the hydrogel composition will likely experience a shear force from the stimulation leads pulling parallel to the skin's surface, as opposed to a tensile force exerted perpendicular to the skin's surface. Lap shear testing was conducted done with substrates of stainless steel, glass, and porcine skin to simulate the human skin during sFES. Since the PA70-TA-PEDOT-CMC sample depicted optimal rheological properties of the examples in Table 1, it was selected as the sample to be sandwiched between substrates for the adhesion test.

The larger error range of adhesive strength to the porcine substrate compared to other substrates may be attributed to differences in water concentration and skin surface conditions of the purchased porcine samples. The hydrogel composition showed a lap shear strength of 2.78±1.28 kPa, two folds lower than the shear adhesive strength of commercial biodegradable fibrin tissue sealants when applied on porcine skin.

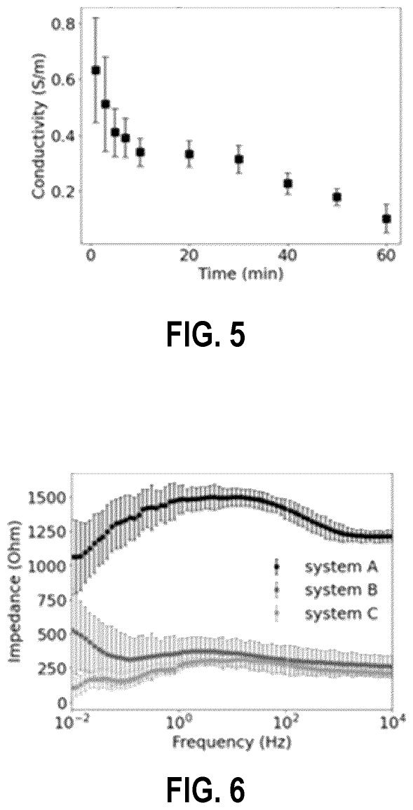

The transient conductivity of the hydrogel composition was next examined. FIG. 5 shows changes in the conductive properties of the PA70-TA-PEDOT-CMC sample within the timeframe of an hour measured immediately after the addition of PA, every two minutes for the first ten minutes, and every ten minutes thereafter. This timeframe was selected because typical sFES sessions last for about 45 minutes. The error bars show the standard deviation of seven samples, with a large deviation in the first ten minutes due to the gelation process taking place as the surface of the material transforms from a viscous gel into a solid hydrogel. Since the sol-gel transition occurred within the first ten minutes, the surface of the pre-gel solution was effectively liquid, resulting in high error bars of measured conductivity initially.

The electrical properties of the hydrogel composition were found to stabilize immediately after gelation, slowly decreasing as water evaporated from the surface of the hydrogel composition when exposed to air. However, in use, it is expected that the bioelectronic interface will be substantially isolated from air, and only the section of hydrogel composition directly in contact with the skin will be responsible for mediating and maintaining a stable and reliable current. Furthermore, although the conductivity showed a steady decline across multiple samples, the overall conductivity was maintained above 50 mS/m in all samples within 1 h, and no change across orders of magnitude is demonstrated, showing that the hydrogel composition gives satisfactory electrical properties in terms of amplitude and stability. However, because PEDOT:PSS is a mixed ionic and electronic conductor, nonlinearities in skin resistance and the change of charge carriers from electronic to ionic across the bioelectronic interface may further influence the ultimate performance during sFES, causing it to deviate from the trends exhibited in material characterizations.

FIG. 6 is a Bode plot of the impedance of various surface stimulation electrodes in contact with porcine trotters. System A was carbon tape with a commercial self-adhesive interface; system B was the PA70-TA-PEDOT-CMC composition twenty minutes after the addition of PA; and system C comprised stainless steel stimulation electrodes with commercial electrolyte gel as the interface, fixed onto the porcine trotter with tape. The error bars show the standard deviation of five samples; for each sample, a new set of porcine trotter, electrode material, and interfacing material was used for the measurements. The high error bars account for biological variances in electrical properties and layer thickness of the skin, fat, and muscle tissues across different porcine trotters. Compared to commercial self-adhesive and flexible electrodes plotted in blue, the performance of the hydrogel composition (system B) drastically lowered the interfacial impedance with the skin, closely matching the performance of metal electrodes and electrolyte gels. This was expected from the high water content in the hydrogel composition electrodes, which mimics the ionic transport of stimulation current in commercial electrolyte gels, as opposed to dry electrodes. Furthermore, since the application of the hydrogel composition effectively fills the crevices on the skin surface, which better mediates the injected current. Thus, the hydrogel composition showed satisfactory performance as sFES electrodes against commercial and conventional methods of sFES delivery.

Use of the Hydrogel Compositions as sFES Electrodes

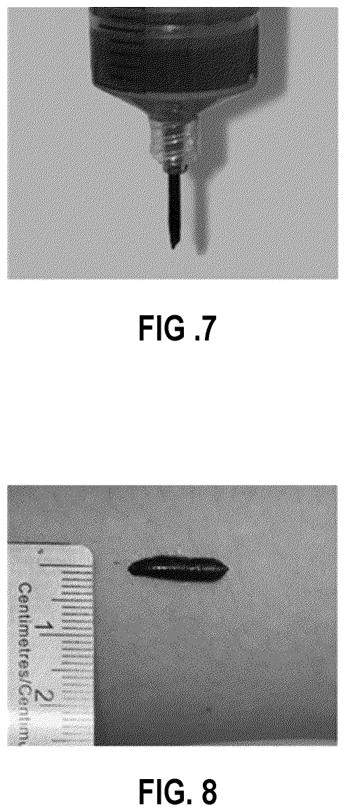

The same composition, PA70-TA-PEDOT-CMC, was compared to commercial self-adhesive sFES electrodes to stimulate eye closer. Immediately after the addition of PA, the pre-gel solution was transferred to a single-barreled syringe with an opening of 1.5 mm in diameter, and extruded. FIG. 7 shows the extruded line of hydrogel composition, with a diameter approximately equal to the opening diameter. FIG. 8 shows the extruded hydrogel composition “painted” on a human arm (e.g., deposited by the syringe). After gelation, the width of the painted line was less than about 3 mm. Other line widths may be achieved by increasing the extrusion rate or the nozzle diameter, but it is considered that the widths shown here, obtainable with an extrusion opening or nozzle from about 2 mm to 20 mm is suitable for sFES applications. For example, the most intricate muscles on the facial region vary from about 29 to about 65 mm. The extrusion opening or nozzle may have different geometries depending on the intended application, such as an elongated or rectangular opening for extruding a ribbon of hydrogel composition.

With the extrusion method described above, the pre-gel solution was first painted onto conductive carbon fibers with wired connections to the stimulator, then painted along the direction of the orbicularis oculi, a sphincter muscle around the upper and lower eyelids with the main function of closing the eyelid. For patients with facial palsy impeding voluntary contractions of the facial muscles, sFES sessions could prevent muscle atrophy, restore muscle volume, and recover lost motor functions. FIG. 8 depicts the comparison of the hydrogel composition with commercial self-adhesive electrodes (MyndMove, MyndTec) in inducing eyelid closure on an able-bodied individual with sFES. Photograph (i) shows the placement of the commercial self-adhesive electrodes to stimulate eye closure according to Ilves, M., Lylykangas, J., Rantanen, V., MAkel, E., Vehkaoja, A., Verho, J., Lekkala, J., Rautiainen, M., Surakka, V., “Facial muscle activations by functional electrical stimulation,” Biomedical Signal Processing and Control 48, 248-254 (2019). Photograph (ii) shows complete sFES-induced eye closure with 3.55 mA.

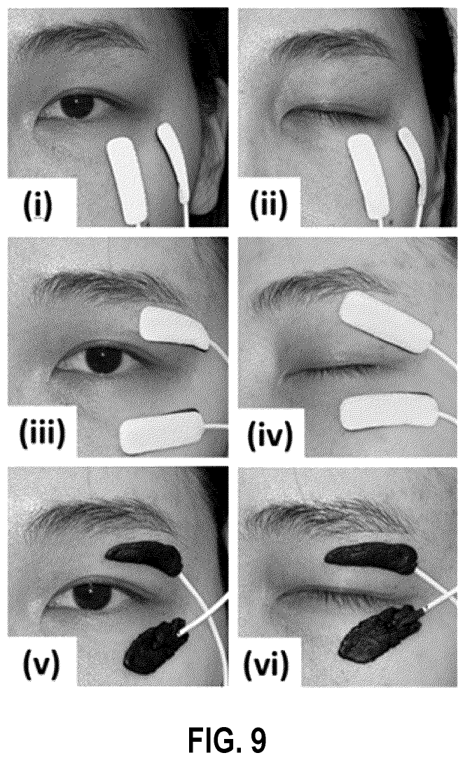

Photographs (iii) and (v) show the placement of the commercial self-adhesive electrodes and the hydrogel composition electrodes, respectively, along the orbicularis oculi muscle. Photographs (iv) and (vi) show complete sFES-induced eye closure using the commercial self-adhesive electrodes and the hydrogel composition electrodes, respectively. Under identical stimulation waveform, the commercial electrodes with fixed dimensions of 1 cm×2 cm required 3.00 mA for full closure, while the hydrogel composition electrodes of area less than 0.7 cm×1.8 cm required 2.85 mA to establish full closure. The subject reported a greater tingling sensation around the eye with the electrode placement locations as shown in FIG. 9 (i) and (iii). Again, without wishing to be bound by theory, This may be attributed to the paintability of the hydrogel composition, enabling a better conforming configuration that is slender and curved around the eye, permitting the sFES electrode to be customized in the direction of the orbicularis oculi muscle growth that is distinct to the subject's unique facial features.

The rapid gelation of the hydrogel composition after mixing the PA or other crosslinking agent with the hydrophilic matrix polymer necessitates the separate storage of the crosslinking agent prior to mixing and application. Thus, the constituents of the hydrogel composition may be provided in a kit or package that facilitates mixing and application of the composition. As mentioned above, the constituents may be provided in two precursors, the second of which comprises the crosslinking agent. The patient or other user would simply mix the two precursors in a suitable container, then use an applicator such as a brush, sponge, or spatula to apply the hydrogel composition to the skin and to the conductor providing an electrical connection to the stimulator or other device. Alternatively, the hydrogel composition may be transferred to a syringe and extruded onto skin or onto the conductor as discussed above.

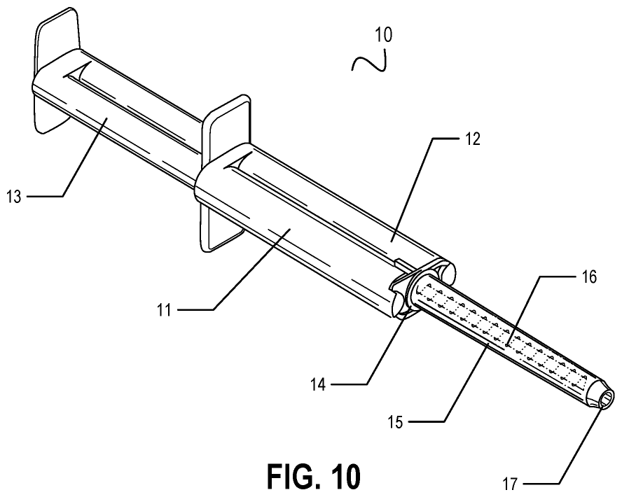

Since transferring the hydrogel composition to a dispensing apparatus may prove challenging to a patient, the precursors may be conveniently packaged in discrete chambers of a handheld dispensing apparatus, such as the two barrels of a double-barred syringe. A simple embodiment of such a syringe 10 as a dispensing apparatus is shown in FIG. 10. Each precursor is stored in one of the barrels 11, 12. The barrels 11, 12 are sealed in an airtight fashion, at one end by the plunger 13 and at the other end with a frangible wall (not shown) blocking aperture 14. To mix and dispense the hydrogel composition, a mixing chamber 15 is mounted to the syringe 10, fracturing the frangible wall. Depressing the plunger 13 causes the two precursors to enter the mixing chamber 15, where they are combined by a static mixture 16 (shown in phantom) prior to extrusion at a nozzle 17. Alternative embodiments will be apparent to those skilled in the art, such as a flexible, squeezable tube with two cavities each containing a precursor.

The examples and embodiments described above thus provide a paintable, self-adhesive, and conductive hydrogel that may be used to create customizable electrodes that can be “painted” or deposited directly on a patient's skin, adhering to both the skin surface and to a conductor or lead. When the hydrogel composition is initially mixed, prior to gelation, it exhibits sufficient viscosity to enable the composition to be shaped into an electrode of the desired size and shape, corresponding to the target muscles and the individual patient's features. The composition then undergoes sol-gel transition within minutes and can provide satisfactory skin adhesion and sufficiently stable conductivity for use in sFES or other therapies. Additionally, the hydrogel composition demonstrates good reduction of interfacial impedance when in contact with skin, potentially enabling a reduction in stimulation amplitude during sFES.

Thus, there is provided a self-adhesive, conductive hydrogel composition for use as a skin-contacting electrode, comprising a crosslinked carboxymethyl cellulose (CMC) hydrogel mixed with an adhesive agent and poly(3,4-ethylenedioxythiophene) polystyrene sulfonate (PEDOT:PSS) as a conductive agent.

In one aspect, the CMC and/or PEDOT:PSS may be crosslinked by phytic acid. The weight ratio of phytic acid (when supplied as 50 wt % in water) to CMC may be about 30% to about 80%, about 65% to about 75%, and more specifically about 70%.

In one aspect, the composition comprises between about 3 and about 5% w/w of PEDOT:PSS, and more specifically between about 3 and about 4.5% w/w of PEDOT:PSS.

In another aspect, the adhesive agent comprises tannic acid, polydopamine, or both tannic acid and polydopamine. In some aspects, the adhesive agent is tannic acid, and the composition comprises between about 3 and about 5% w/w of tannic acid, and more specifically between about 4.0 and about 4.2% w/w of tannic acid. The same ranges may be employed when polydopamine is used as the adhesive agent. In some implementations, where the adhesive agent comprises both tannic acid and polydopamine, the composition may comprise between about 3 and about 5% w/w of tannic acid and about 1.6 and about 10% w/w of polydopamine.

In one aspect, the phytic acid may serve as a conductive agent as well.

In another aspect, a self-adhesive, conductive composition suitable for application to human skin for use as an electrode in surface functional electrical stimulation, comprises a hydrogel comprising a crosslinked hydrophilic matrix polymer; up to about 5% w/w of a conductive agent; and up to about 5% w/w of an adhesive agent. The crosslinked hydrophilic matrix polymer comprises a polysaccharide and a crosslinking agent. The constituents of the composition may comprise the components in the proportions set out above.

There is also provided a method of preparing a self-adhesive, conductive composition suitable for use as a skin-contacting electrode, the method comprising: mixing a hydrophilic matrix polymer or a salt thereof with water to provide a hydrogel solution; mixing the hydrogel solution with an adhesive agent and a conductive agent to provide a precursor; and mixing the precursor with a crosslinking agent. In some aspects, the hydrophilic matrix polymer comprises a polysaccharide, such as CMC, and the other constituents of the composition may comprise the components in the proportions set out above. The hydrogel solution may be provided by mixing sodium carboxymethyl cellulose and water, and heating the hydrogel solution to about 60° C. prior to mixing the adhesive agent with the hydrogel solution. The PEDOT:PSS may be provided as a dispersion in water. In some implementations, the conductive agent is mixed with the hydrogel solution after the adhesive agent is mixed with the hydrogel solution, the method further comprising cooling the mixture of the hydrogel solution with the adhesive agent to about room temperature prior to mixing the conductive agent with the hydrogel solution. The precursor may be cooled to about 4° C. prior to mixing the precursor with the crosslinking agent.

There is also provided a method of applying the composition described herein as a skin-contacting electrode, the method comprising: coating a conductor with the composition; applying the composition to a skin surface; and adhering the coated conductor to the composition applied to the skin surface. The composition coating the conductor may be permitted to harden prior to applying the composition to the skin surface.

In some implementations, applying the composition to the skin surface comprises dispensing the composition directly on the skin surface using a dispensing apparatus, or alternatively painting or spreading the composition directly on the skin surface.

The composition may be provided in a self-adhesive electrode suitable for use on human skin, optionally shaped according to a target region on a patient, for example determined by the intended location of one or more muscles of the patient.

There is also provided a kit for dispensing a self-adhesive, conductive composition for use as a skin-contacting electrode, the kit comprising: a first precursor comprising a hydrophilic polymer hydrogel mixture; a second precursor comprising a crosslinker; either the first precursor or the second precursor comprising a conductive agent; and either the first precursor or the second precursor comprising an adhesive agent.

The kit may further include a dispensing apparatus for combining the first precursor and the second precursor. In one aspect, the dispensing apparatus comprises discrete chambers containing the first precursor and the second precursor, both discrete chambers in fluid communication with a mixing chamber and a dispensing nozzle having an appropriate shape and size. The mixing chamber may comprise a static mixer. The first precursor may include both the conductive and adhesive agents, premixed with the hydrogel.

In the foregoing, the constituents may be provided commercially in powder or dispersed in water, as the case may be. The composition may comprise CMC up to about 30%, and specifically about 7% w/w to water; tannic acid up to about 285%, and specifically about 50% w/w to water; polydopamine up to about 70% w/w, and specifically about 1.5% w/w to CMC. The composition may comprise up to 100%, and specifically about 50% w/w to water of PEDOT:PSS, supplied as 1 wt. % in water, and phytic acid in a range of about 30% to about 80%, and specifically about 70% w/w to CMC, where the phytic acid is supplied at 50 wt. % in water. “Up to about” may be considered as starting above zero, e.g. 0.1%.

It should be understood that this description is not intended to be limiting, and that the examples contemplated herein include all alternatives, modifications, and equivalents as would be appreciated by the person skilled in the art, and are not meant to limit the scope of the subject matter recited herein. For example, some steps or acts in a process or method may be reordered or omitted. Although the features and elements various examples or embodiments may be described as being in particular combinations, the person of ordinary skill in the art will appreciate which features or elements can be used alone, without the other features and elements of the embodiments, or in various combinations with or without other features and elements disclosed herein. Further, individual features or variations described in respect of one example or embodiment in this disclosure may be used with other examples or embodiments mentioned herein, provided separately, or in any suitable subcombination, as would be understood by the person skilled in the art.

1. A self-adhesive, conductive hydrogel composition for use as a skin-contacting electrode, comprising a crosslinked carboxymethyl cellulose (CMC) hydrogel mixed with an adhesive agent and poly(3,4-ethylenedioxythiophene) polystyrene sulfonate (PEDOT:PSS) as a conductive agent.

2. The composition of claim 1, wherein the CMC and/or PEDOT:PSS is crosslinked by phytic acid.

3. The composition of claim 2, wherein the weight ratio of phytic acid (50 wt % in water) to CMC is about 30% to about 80%.

4. The composition of claim 3, wherein the weight ratio of phytic acid (50 wt % in water) to CMC is about 70%.

5. The composition of claim 1, comprising between about 3 and about 5% w/w of PEDOT:PSS.

6. The composition of claim 5, comprising between about 3 and about 4.5% w/w of PEDOT:PSS.

7. The composition of claim 1, wherein the adhesive agent comprises tannic acid, polydopamine, or both tannic acid and polydopamine.

8. The composition of claim 7, wherein the adhesive agent comprises tannic acid.

9. The composition of claim 8, comprising between about 3 and about 5% w/w of tannic acid.

10. The composition of claim 9, comprising between about 4.0 and about 4.2% w/w of tannic acid.

11. The composition of claim 7, wherein the adhesive agent comprises polydopamine.

12. The composition of claim 11, comprising between about 3 and about 5% w/w of polydopamine.

13. The composition of claim 12, comprising between about 4.0 and about 4.2% w/w of polydopamine.

14. The composition of claim 7, wherein the adhesive agent comprises both tannic acid and polydopamine.

15. The composition of claim 14, comprising between about 3 and about 5% w/w of tannic acid and about 1.6 and about 10% w/w of polydopamine.

16. The composition of claim 1, wherein the self-adhesive, conductive composition is suitable for use in surface functional electrical stimulation.

17. The composition of claim 1, further comprising phytic acid as a conductive agent.

18-54. (canceled)

55. A self-adhesive electrode suitable for use on human skin comprising a deposit of the composition of claim 1.

56. The electrode of claim 55, further comprising a lead embedded within the deposit.

57-81. (canceled)
